(19) United States
(12) Patent Application Publication
(10) Pub. No.: US 2018/0104729 A1
(43) Pub. Date: Apr. 19, 2018

(54) AUDITABLE INFECTIOUS AND HAZARDOUS WASTE DISPOSAL

(71) Applicant: AEMERGE LLC, Fortville, IN (US)

(72) Inventor: Landon C.G. Miller, Fortville, IN (US)

(21) Appl. No.: 15/292,516

(22) Filed: Oct. 13, 2016

(51) Int. Cl. B09B 3/00; A61L 2/00; A61L 2/04; B02C 19/00

(52) U.S. Cl. CPC B09B 3/0075; B09B 3/0083; H04L 67/06; A61L 2/04; B02C 19/0075; A61L 2/0023

(57) ABSTRACT

A system for auditable treatment and destruction of waste is provided that includes a computer server with a database connected to a network. Readers and sensors record the set of containers from inventory into a waste processing line via a belt conveyor running from an exterior of a sealed enclosure to a shredder. An oxidizer is in fluid communication with the sealed enclosure. A feed conveyor is provided for transfer of shredded material from the shredder to a carbonizer, Temperature data and data of the electrical load and/or speed data are communicated to the process control computer and then to the computer server. A process for creating an audit report of the destruction is also provided.

### FIELD OF THE INVENTION

The present invention in general relates to a system for treating infectious waste; and in particular to an automated tracking and recording system for medical and hazardous waste handling and disposal that provides auditable information regarding the disposition of the inputted waste.

### BACKGROUND OF THE INVENTION

Infectious medical waste is generated in the research, diagnosis, treatment, or immunization of human beings or animals and has been, or is likely to have been contaminated by organisms capable of causing disease. Infectious medical waste includes items such as: cultures and stocks of microorganisms and biologicals; blood and blood products; pathological wastes; radiological contrast agents, syringe needles; animal carcasses, body parts, bedding and related wastes; isolation wastes; any residue resulting from a spill cleanup; and any waste mixed with or contaminated by infectious medical waste. Facilities which generate infectious medical waste include: hospitals, doctors' offices, dentists, clinics, laboratories, research facilities, veterinarians, ambulance squads, and emergency medical service providers, etc. Infectious medical waste is even generated in homes by home health care providers and individuals, such as diabetics, who receive injections at home.

Before infectious medical waste can be disposed of the waste must be sterilized. Traditional sterilization methods include: incineration; steam treatment or autoclaving; and liquid waste may be disposed of in approved sanitary sewers. More recent methods that have been developed include microwave irradiation and use of various chemical washes.

Transforming waste from a liability to an asset is a high global priority. Currently employed technologies that rely on incineration to dispose of carbonaceous waste with useable quantities of heat being generated while requiring scrubbers and other pollution controls to limit gaseous and particulate pollutants from entering the environment. Incomplete combustion associated with conventional incinerators and the complexities of operation in compliance with regulatory requirements often mean that waste which would otherwise have value through processing is instead sent to a landfill or incinerated off-site at considerable expense. As medical waste often contains appreciable quantities of synthetic polymers including polyvinyl chloride (PVC), incineration of medical waste is often accompanied by release of chlorine, ClOₓ, SOₓ, and NOₓ air pollutants that must be scrubbed from the emitted gases. Alternatives to incineration have met with limited success owing to complexity of design and operation outweighing the value of the byproducts from waste streams.

While there have been many advances in the treatment and disposal of infectious waste, there still exists a need for automated systems and methods for tracking, recording, and reporting in an auditable format the safe collection, transfer, and treatment of infectious and hazardous waste that maximize the economic return from the treated waste while enhancing handling safety, client and regulatory reporting, and environmental controls.

### SUMMARY OF THE INVENTION

A system for auditable treatment and destruction of hazardous and infectious waste is provided that includes a computer server with a database connected to a network. A first reader connected via the network to the computer server is provided to record identifying information about a set of containers into inventory. The set of containers holding hazardous and infectious waste delivered for disposal. A waste processing line has a process control computer that controls the waste processing line and is connected to the network. A second reader electrical in communication with the process control computer is present on the line to record the set of containers as the containers are moved from inventory into the waste processing line. The line has a shredder within a sealed enclosure. A belt conveyor running from an exterior of the sealed enclosure to the shredder supplies the set of containers for processing. An oxidizer is in fluid communication with the sealed enclosure and adapted to destroy airborne infectious matter from the sealed enclosure. A feed conveyor is provided for transfer of shredded material from the shredder to a carbonizer, the carbonizer having a chain belt to move shredded material therethrough. At least one temperature sensor of the temperature of the carbonizer and at least one motor controller provide the electrical data needed to calculate the speed of the carbonizer chain belt are present and transmit the temperature data and data of the electrical load and/or speed data to the process control computer and to the computer server.

A process for creating an audit report includes a time fence being defined that designates an allowable processing window. A log of processing variables is created that is updated in defined intervals during the time fence. A message is formatted with the log of processing variables during the time fence. The message is then sent over the network as an audit report.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a block diagram of an overall system for auditable infectious waste treatment according to an embodiment of the invention;

FIG. 2 is a block diagram of an infectious waste treatment system according to an embodiment of the invention;

FIG. 3 is a side section view depicting an encapsulated shredding and infectious matter escape prevention sub-system according to an embodiment of the invention;

FIG. 4 is an oxidizer adapted for use with embodiments of the invention; and

FIG. 5 is a block diagram of a top loaded infectious waste treatment system according to an embodiment of the invention.

### DESCRIPTION OF THE INVENTION

The present invention has utility as an automated system and method for tracking, recording, and reporting in an auditable format the safe collection, transfer, and treatment of infectious and hazardous waste that maximizes the economic return from the treated waste while enhancing handling safety, environmental controls, and client and regulatory reporting. Embodiments of the invention track and record information for received infectious and hazardous waste, and provide an audit trail for the handling and processing of the waste, as well as the final disposition of any remaining waste that has no further processable economic value and hence is to be placed in a landfill. Electronic data transfer protocols are used to transfer information to a database that provides an audit trail of the infectious and hazardous waste that is being treated and disposed of. Based on the recorded data and a successful disposition of the treated infectious and hazardous waste a “certificate of destruction” may be issued.

Embodiments of the automated system for auditing infectious and hazardous waste treatment and disposal may use a medical waste handling and shredding sub-system, as disclosed in co-pending applications PCT/US16/13067 “Infectious Waste Disposal” filed Jan. 12, 2016 and PCT/US16/22061 “Integrated Collection of Infectious Waste and Disposal Thereof” filed Mar. 11, 2016 both of which are included by reference in their entirety herein, that feeds partially processed waste to an oxidizer to eliminate potential airborne infectious waste prior to transforming the medical waste into useful co-products. In accordance with the present invention, medical waste is transformed into value added products including hydrocarbon based gases, hydrocarbon-based liquids, carbonized material, and recovered precious metals and rare earth materials in a system having as its transformative element an anerobic, negative pressure, or carbonization system. With medical waste as a feedstock for the production of valuable products, the present invention provides an economically viable and environmentally more responsible alternative to traditional methods of medical waste treatment.

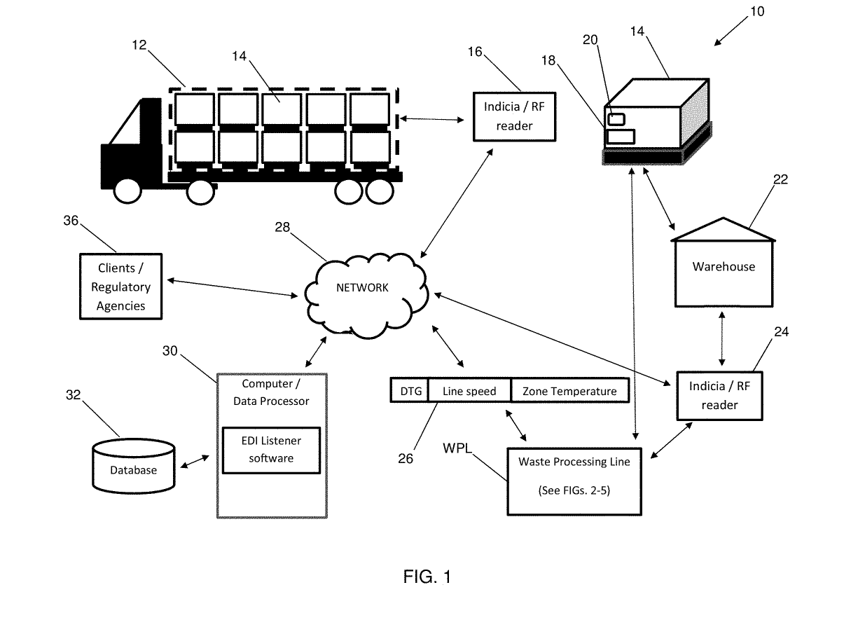

Referring now to the figures, embodiments of an inventive infectious auditable waste system are described. FIG. 1 is a block diagram of an overall system 10 for auditable infectious waste treatment. As shown in FIG. 1 a truck 12 brings a load of infectious or hazardous waste in a collection of waste containers 14. The waste containers 14 are stacked in the truck 12, and may be drums, boxes, or palletized box containers that are commonly known as gaylords. Each of the individual containers 14 may be identified with at least one of a machine readable indicia 18 or a radio frequency identification tag 20 (RFID). The machine readable indicia 18 may illustratively include barcodes and quick response (QR) codes. Upon delivery of the waste to be processed, the indicia 18 are read or the RFID 20 are scanned with the reader 16. If the containers 14 are coded with RFID tags 20, the truck may be driven through an overhead gantry that holds the reader 16 to read the contents of the truck. The scanned containers 14 of waste may be placed in a warehouse 22 as inventory or sent directly to a waste processing line (WPL). If the waste is warehoused, the containers 14 are rescanned with reader 24 as the containers of waste are removed from inventory and introduced to the waste processing line (WPL). The scanned identifying information from the containers 14 are sent via a network 28 to a computer server 30 that maintains a database 32. In a specific embodiment the database 32 is based on enterprise resource planning (ERP), which is a category of business-management software—typically a suite of integrated applications—that an organization can use to collect, store, manage and interpret data from many business activities, including: product planning, purchasing, manufacturing, or service delivery.

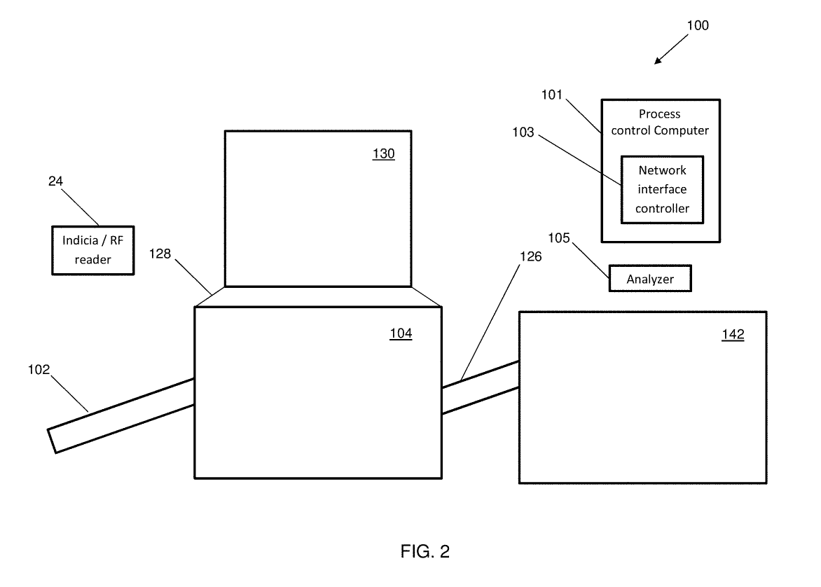

Continuing with FIG. 1, the waste is processed using a waste processing line (WPL) that is described in further detail in FIGS. 2-5. Processing may be tracked in units of time referred to as a “time fence” which is an allowable processing window. A process control computer system 101 in FIG. 2 produces a log of various processing parameters. Processing parameters may illustratively include derivative thermogravimetric (DTG), conveyor line speed, and carbonizer temperature by zone. Thermalgravimetric analysis (TGA) is a method of thermal analysis in which changes in physical and chemical properties of materials are measured as a function of increasing temperature (with constant heating rate), or as a function of time (with constant temperature and/or constant mass loss). TGA can provide information about physical phenomena, such as second-order phase transitions, including vaporization, sublimation, absorption, adsorption, and desorption. Likewise, TGA can provide information about chemical phenomena including chemisorptions, desolvation (especially dehydration), decomposition, and solid-gas reactions (e.g., oxidation or reduction). TGA may be used to determine selected characteristics of materials that exhibit either mass loss or gain due to decomposition, oxidation, or loss of volatiles. The analysis may be conducted with analyzer 105 with the information sent with the network interface controller 103 via network 28. The network 28 may be a local area network (LAN), wide area network (WAN), or the Internet. Information may be sent via wired or wireless mediums. In a specific inventive embodiment, the collected information from the auditable waste processing system is in a standardized format that allows for electronic data interchange (EDI). EDI allows computer to computer information transfer without human intervention. The EDI listener 34 shown in the computer server 30 “listens” for EDI protocols and accepts data that is sent in acceptable formats to be included in the database 32. Waste process information may be retrieved from the database 32 by the computer 30 to generate reports and conduct audits that are made available to clients and regulatory agencies 36.

FIG. 2 is a block diagram of an infectious waste treatment system 100 according to an embodiment of the invention. An encapsulated shredding and infectious matter escape prevention sub-system 104 encloses a shredder in a negative pressure sealed environment that acts to contain residue and contaminants from escaping into the environment during the shredding operation. The infectious waste is loaded into the sub-system 104 via belt conveyor 102. The belt conveyor 102 introduces the infectious or contaminated waste in bags or containers, that are scanned with reader 24 as the containers of waste are introduced into the subsystem 104. An oxidizer 130 destroys any airborne infectious matter that exits through hood 128 at the top of the sub-system 104.

As used herein an oxidizer is defined to also include a thermal oxidizer and catalytic oxidizer; such systems are commercially available and in widespread usage.

Feed conveyor 126 transfers the shredded material from the sub-system 104 to the carbonizer 142. It is appreciated that feed conveyor 126 also includes augers, shuttle bins, and other conventional devices to transit shredded material. The analyzer 105 may be used to analyze the outputted waste, illustratively including thermogravimetric analysis (TGA). Physical samples—aliquots of the outputted treated waste may be taken, packaged and labeled with lot information, and saved by the analyzer 105. The process control computer 101 controls the operating parameters of the system 100, and the network interface 103 provides formatted information to on the network 28.

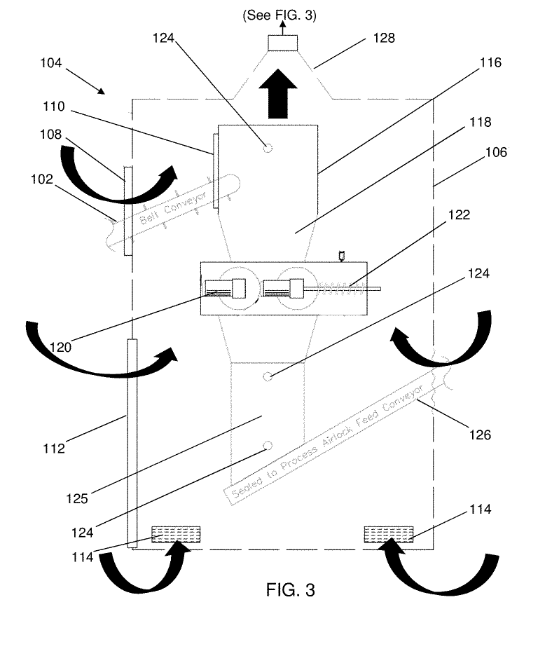

FIG. 3 is a side section view depicting the encapsulated shredding and infectious matter escape prevention sub-system 104. The dotted lines represent the containment walls 106 that enclose the shredder 116. The enclosure of the sub-system 104 is maintained at a negative pressure to draw in air (as opposed to expelling air) as represented by the arrows into the vents 114, as well as into the exterior flap 108 that permits containerized waste to enter the sub-system 104 via the belt conveyor 102, and other openings such as for the feed conveyor 126 and service door 112. The exterior flap 108 is readily formed of rubberized materials, polymeric sheeting, as well as metals. Service door 112 is provided in some inventive embodiments to allow service workers to enter the enclosure. It is appreciated that a service person may be required to wear protective clothing and a filter mask. In a specific embodiment the service door 112 may be a double door airlock, where only one door is open at a time to minimize the escape of contaminants into the environment. In still other embodiments, the air handling system modifies operation during opening of the service door 112 to maintain a negative pressure during opening to inhibit airborne escape of potential pathogens. Hopper flap 110 acts to allow containerized waste to enter the hopper 118 of the shredder 116, while also acting as a seal around the belt conveyor 102. The hopper flap 110 is readily formed of rubberized materials, polymeric sheeting, as well as metals. At the bottom of the hopper 118, an auger 122 that is driven by one or more motors 120 shreds the waste. In an embodiment the motors 120 may be variable frequency drive (VFD) motors. The shredded material is accumulated in a process airlock 125 that supplies material to a feed conveyor 126. Levels and presence of material within the hopper 118 and the process airlock 125 are controlled via sensors 124. In a specific embodiment the sensors 124 are through beam sensors (TBS). Feed conveyor 126 is sealed to the process airlock 125, and transports the shredded material from the sub-system 104 to the carbonizer 142. Hood 128 collects airborne contaminants for introduction into the oxidizer (TO) 130.

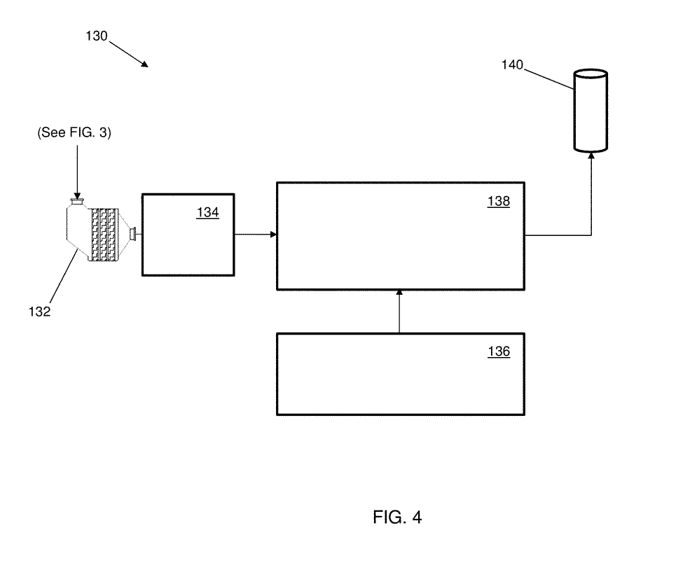

FIG. 4 is a block diagram of an oxidizer 130 adapted for use with embodiments of the invention that acts as a fume incinerator for the containment room of sub-system 104. Large particle screener 132 filters out particles from the exhaust stream of airborne contaminants. A filter differential sensor may be employed to detect when a filter is clogged and requires replacement. A blower 134 draws in the exhaust stream and blows the exhaust stream into the combustion tube 138. A gas supply 136 supplies fuel for burners in the combustion tube 138. In specific embodiments the oxidizer 130 is run on a mixture of natural gas and reaction-produced carbonization process gases re-circulated to transform the heat through the use of either conventional steam boilers or to Organic Rankin Cycle strategies to operate electrical turbine generators, or in the alternative, to reciprocating engine driven generators, and thereby generate the heat needed to produce power while also operating the carbonization process in the carbonizer 142. This heat capture produces more waste heat than is used to heat water and generate steam for turbines or steam reciprocating engines. This heat in some inventive embodiments is used to preheat feedstock or for other larger process purposes. The pre-processing heating system preheats feedstock material prior to entering the reactor tube to both reduce moisture and improve overall system yield. Roof exhaust stack 140 vents cleaned exhaust to the environment.

An apparatus for anaerobic thermal transformation processing as carbonizer 142 to convert waste into bio-gas; bio-oil; carbonized materials; non-organic ash is detailed in U.S. Pat. No. 8,801,904; the contents of which are incorporated herein by reference.

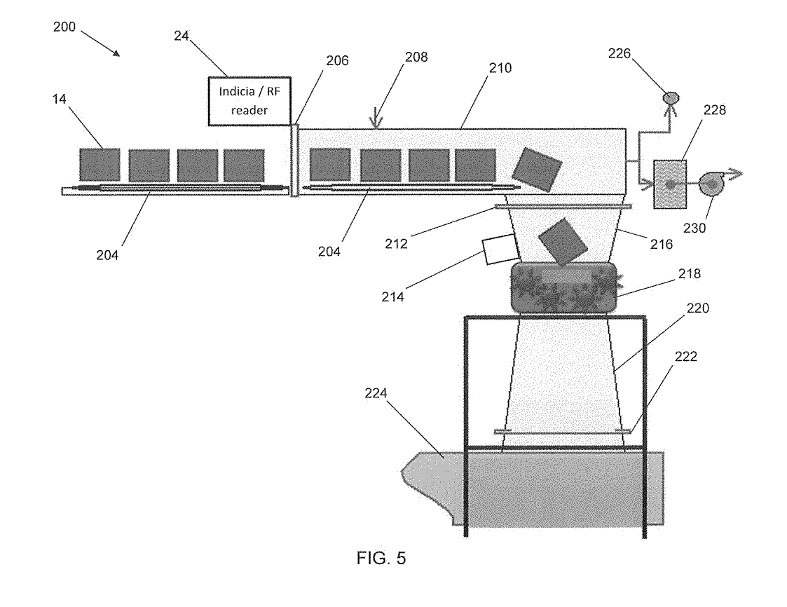

FIG. 5 illustrates a block diagram of a shredder feed system 200 for treatment and recovery of usable products from waste feedstock illustratively including medical and infectious waste, where the carbonizer 142 is that described with respect to the aforementioned drawings. The feed system 200 utilizes conveyers 204 to feed and transport containers 14 of waste into and through the pre-shred air-lock tunnel 210 and into a shred feed hopper 216. The reader 24 reads the indicia or RFID tag on each of the containers 14 prior to entry into the pre-shred air-lock tunnel 210. The pre-shred air-lock tunnel 210 has an airtight open and close inlet valve (door) 206 and an outlet valve (door) 212 to the shred feed hopper 216. The pre-shred air-lock tunnel 210 may have nitrogen inputted at valve 208 to provide an inert atmosphere in the air-lock tunnel 210. In a specific embodiment the waste may be treated with a wet scrubber 214. Medical waste that contains appreciable quantities of synthetic polymers including polyvinyl chloride (PVC), when incinerated is often accompanied by release of chlorine, ClOₓ, SOₓ, and NOₓ air pollutants that are preferably scrubbed from the emitted gases to limit air pollution. The wet scrubber 214 facilitates a reaction with chloride gas to yield a resultant hydrochloric acid (HCl) product. In order to withstand corrosion caused by HCl, and other byproducts produced in operation of an inventive system, system components are readily formed of solid-solution-strengthened, high-temperature corrosion-resistant alloys that are generally rich in nickel and chromium/cobalt as major constituents with illustratively include 37Ni-29Co-28Cr-2Fe-2.75Si-0.5Mn-0.5Ti-0.05C-1W-1Mo-1Cb, S13Cr, 316L (S31603), 22 Cr duplex, 25 Cr duplex, 28 (N08028), 825 (N08825), 2550 (N06975), 625 (N06625) C-276 (N10276), where parentheticals correspond to the UNS numbers for a particular alloy. These alloys are resistant to the effects of HCl may be used in the construction of one or more of the wet scrubber 214, shred feed hopper 216, shredder 218, and other components of the system 200 that may contact the corrosive HCl and chlorine, such as the sealed enclosure, the shredder, the belt conveyor, the oxidizer, or the feed conveyor.

Continuing with FIG. 5, the shredder 218 may be a two or four shaft shredder that is mounted so that all shredded waste material and liquids exit the bottom of the shredder 218 into a collection hopper 220 that meters and distributes the waste with a post-shred air-lock 222 directly into a carbonizer 142. It is appreciated, precious metals and rare-earth materials for example associated with medical imaging may be obtained by burning off the carbon product to obtain carbon dioxide and the resultant metal materials. For example, contrast agents used for radiological procedures are a source of precious metals and rare earths. Gasses from the air-lock tunnel are managed with an oxygen sensor 226 and escaping particulate is filtered with a high-efficiency particulate air (HEPA) filter 228, and is the expelled through a blower 230 to an oxidizer illustratively including a thermal oxidizer.

### EXAMPLES

### Example 1-Transfer Station to Auditable Treatment Facility System—Palletized Waste

A truck 12 as described with respect to an embodiment of FIG. 1 is configured to hold a lot of forty-eight double stacked pallets 14 of waste that are each labeled with the lot number and container number are scanned into processable inventory with identifying data stored in an ERP related database. Subsequently, the lot of containers 14 are scanned out of inventory and into a carbonizer processor as described in FIGS. 2-5. A “time fence” is used to define an allowable processing window, the processing of the waste controlled by the process control computer 101. A log is maintained during the processing window of DTG, carbonizer temperature by zone, and dragchain motor speed. The log is updated with a new record in one minute intervals, with a message formatted as an EDI of other format sent over the network 28 every fifteen minutes that is composed of the fifteen individual minute records.

### Example 2-Audited Production of Certificate of Destruction Generation

When a scanned in lot consisting of all waste containers 14 has been checked out of inventory and into a carbonizer for waste processing, and a “time fence” cross check shows that all containers 14 have been subjected to the carbonizer process then a Certificate of Destruction is produced for that lot. The Certificate of Destruction may be provided to clients and regulatory agencies 36.

As a person skilled in the art will recognize from the previous detailed description and from the figures and claims, modifications and changes can be made to the preferred embodiments of the invention without departing from the scope of this invention defined in the following claims.

1. A system for auditable treatment and destruction of hazardous and infectious waste, the system comprising:
a computer server with a database connected to a network;
a first reader to record identifying information about a set of containers into inventory, said set of containers holding hazardous and infectious waste delivered for disposal, said first reader connected via said network to said computer server;
a waste processing line comprising:
a process control computer that controls said waste processing line and is connected to said network;
a second reader to record said set of containers as said set of containers containers are moved from inventory into said waste processing line, said second reader in electrical communication with said process control computer;
a sealed enclosure;
a shredder within said sealed enclosure;
a belt conveyor to supply said set of containers, said belt conveyor running from an exterior of said sealed enclosure to said shredder;
an oxidizer in fluid communication with said sealed enclosure adapted to destroy airborne infectious matter from said sealed enclosure;
a feed conveyor for transfer of shredded material from said shredder to a carbonizer, said carbonizer having a chain belt to move shredded material through said carbonizer; and
at least one temperature sensor and at least one motor controller, said sensor providing temperature of the carbonizer, and said controller providing the electrical data needed to calculate the speed of the carbonizer chain belt; and
transmission of the temperature data and of the electrical load and/or speed data to the process control computer and then to the computer server.

2. The system of claim 1 wherein said set of containers further comprise machine readable indicia as the identifying information that are scanned by said first and said second reader.

3. The system of claim 1 wherein said set of containers further comprise radio frequency identification tags as the identifying information that are scanned by said first and said second reader.

4. The system of claim 1 wherein said database is based on enterprise resource planning (ERP).

5. The system of claim 1 wherein said process control computer produces a log of processing parameters, said processing parameters comprising results of thermogravimetric analysis (TGA), line speed of said chain belt, and carbonizer temperature by zone.

6. The system of claim 5 wherein said log of processing parameters are sent over the network in a standardized format.

7. The system of claim 6 wherein said standardized format is adapted for electronic data interchange (EDI).

8. The system of claim 7 wherein said computer server runs EDI listener software.

9. The system of claim 1 wherein said computer server generates reports and conducts audits that are made available to clients and regulatory agencies over said network.

10. The system of claim 1 wherein the sealed enclosure is maintained at a negative pressure.

11. The system of claim 1 further comprising a rubberized exterior flap that permits containerized and bagged waste to enter the sealed enclosure via said belt conveyor.

12. The system of claim 1 wherein said sealed enclosure further comprises a hood that collects said airborne contaminants for introduction into said thermal oxidizer.

13. The system of claim 1 wherein said oxidizer further comprises a large particle screener.

14. The system of claim 1 wherein said oxidizer further comprises a blower for that draws in said airborne infectious matter into a combustion tube.

15. The system of claim 1 wherein said oxidizer is a thermal oxidizer.

16. The system of claim 1 wherein said oxidizer further comprises a gas supply that supplies fuel for burners in a combustion tube.

17. The system of claim 16 wherein said oxidizer is run on a mixture of natural gas and reaction-produced carbonization process gases re-circulated to transform heat through the use of a steam boiler, an organic Rankin Cycle, or a combination thereof.

18. The system of claim 1 wherein said shredder further comprises a hopper to receive waste and a process airlock where shredded wasted material accumulates and is transferred to said feed conveyor.

19. A process for creating an audit report for treatment and destruction of hazardous and infectious waste using the system of claim 1, the process comprising:
defining a time fence that designates an allowable processing window for waste containers;
creating a log of processing variables that is updated in defined intervals during the time fence;
formatting a message with the log of processing variables during the time fence;
treating and destroying the hazardous and infectious waste; and
sending the message over the network as an audit report.

20. The process of claim 19 further comprising issuing an audited certificate of destruction when a time fence cross check shows that all containers have been subjected to the carbonizer.
